(19) United States
(12) Patent Application Publication
(10) Pub. No.: US 2026/0071939 A1
(43) Pub. Date: Mar. 12, 2026

(54) ANALYZER FOR MEASURING A CONCENTRATION, COMPRISING A VALVE AND A FILTER

(71) Applicant: Dräger Safety AG & Co. KGaA, Lübeck (DE)

(72) Inventors: Malte Baesler, Lübeck (DE); Tobias Reier, Lübeck (DE); Luis Brunswick Franco, Lübeck (DE); Roberto Da Silva Cirolini, Lübeck (DE)

(21) Appl. No.: 19/320,202

(22) Filed: Sep. 5, 2025

(30) Foreign Application Priority Data
Sep. 6, 2024 (DE) 102024125707.5

(51) Int. Cl. G01N 1/22; G01N 1/24; G01N 33/497

(52) U.S. Cl. CPC G01N 1/2205; G01N 1/24; G01N 2001/241; G01N 33/4972

(57) ABSTRACT

The disclosure relates to an analyzer which is configured to analyze a gas mixture for a specified substance. A tubular input unit can be connected to a main body. A gas mixture can be introduced into the input unit and flows from an inlet to an outlet. A fluid guide unit connects a suction opening in the input unit to a measurement chamber. A suction unit can suck in a gas sample from the input unit by suction and can convey it through the fluid guide unit into the measurement chamber. A sensor can measure the concentration of the substance in a gas sample located in the measurement chamber. A valve optionally opens or closes the fluid guide unit. An electrostatically charged and/or mechanically acting filter in the fluid guide unit is located between the suction opening and the valve.

### CROSS-REFERENCE TO RELATED APPLICATIONS

This application claims the priority of German Patent Application No. 102024125707.5, filed on Sep. 6, 2024, and titled “ANALYZER FOR MEASURING A CONCENTRATION, COMPRISING A VALVE AND A FILTER”, which is hereby incorporated by reference in its entirety for all non-limiting purposes.

### BACKGROUND

The disclosure relates to an analyzer which can analyze a gas mixture for a specified substance and can, in doing so, measure the concentration of the substance in the gas mixture.

### SUMMARY

In one application, the gas mixture is a breath sample exhaled by a test subject, and the substance is breath alcohol or some other substance that can be detected in a breath sample from a test subject. In this application, the aim is to check whether the test subject has consumed alcohol or the other substance, and therefore whether or not the substance is present in the test subject's body.

One aspect of the disclosure is to provide an analyzer which can measure the concentration of a substance in a gas mixture and which has a higher reliability than known analyzers, even if used for a relatively long time period.

This can be achieved by an analyzer having the features of claim 1. Advantageous embodiments are specified in the dependent claims.

The analyzer according to the disclosure can analyze a gas mixture for a specified substance, in particular a breath sample for breath alcohol.

The analyzer comprises a main body and an input unit. The input unit is connected to the main body or can be connected to the main body. In a first implementation, the input unit is permanently connected to the main body. In a second implementation, the input unit comprises a mouthpiece and a tube, with the mouthpiece being detachably connected to the tube and the tube being permanently connected to the main body. In a third implementation, the entire input unit is detachably connected to the main body, i.e. it can be separated as a whole from the main body. In many cases, the second and the third implementation make it easier to comply with hygiene requirements.

The input unit has the shape of a tube, in particular the shape of a cylinder or a truncated cone or a prism with an n-sided cross-sectional area, n>=3. The input unit has an inlet, an outlet, and a lateral surface (surface area). The lateral surface extends between the inlet and the outlet. In one embodiment, the input unit tapers from the inlet toward the outlet. A gas mixture to be analyzed can be introduced into the input unit through the inlet. The introduced gas mixture flows through the input unit toward the outlet. The introduced gas mixture, or at least a portion of the gas mixture that is not drawn off by suction, flows back out of the input unit through the outlet.

A sensor arrangement is located in the interior of the main body. The sensor arrangement comprises a measurement chamber and a sensor. The measurement chamber can receive a gas sample to be analyzed.

A suction opening is located in the lateral surface of the input unit. The suction opening is located at a distance from the inlet and at a distance from the outlet. A fluid guide unit connects an opening in the main body to the measurement chamber. This therefore creates the following situation if the input unit is connected to the main body: The fluid guide unit connects the suction opening in the input unit to the measurement chamber. A gas sample can flow from the input unit through this fluid guide unit into the measurement chamber. The fluid guide unit is located in the interior of the main body. If the input unit is not connected to the main body, the fluid guide unit thus connects an opening in the main body to the measurement chamber.

A fluid guide unit will be understood to mean a component which can guide a fluid, here a gas sample, along a trajectory, this trajectory being predefined by the design and arrangement of the component. A tube and a hose are two examples of a fluid guide unit.

A suction unit in the interior of the main body can carry out the following steps: The suction unit sucks in a gas sample from a gas mixture flowing through the input unit, i.e. branches off the gas sample from the gas mixture. The gas sample is therefore a portion of the gas mixture flowing through the input unit. The remainder of the gas mixture is not drawn off by suction, but instead remains in the input unit and flows out of the input unit through the outlet. The suction unit conveys the gas sample through the suction opening and the fluid guide unit into the measurement chamber. The suction unit can additionally flush out the measurement chamber.

The sensor of the sensor arrangement can measure the concentration (the proportion, the share) and/or the amount, e.g. the weight, of the sought substance in a gas sample while this gas sample is located in the measurement chamber. Usually, the sensor measures a detection variable that correlates with the concentration and/or amount of the substance in the gas sample. A signal-processing evaluation unit of the sensor arrangement applies a predefined functional relationship to the measured detection variable (more precisely: to at least one measured value of the detection variable) and thus derives the concentration and/or amount of the substance in the gas sample. Optionally, the functional relationship depends on multiple measured variables.

The wording is used that a sensor can measure a physical variable, for example the concentration of a substance in a gas sample. This wording means that the sensor can measure the physical variable directly, or at least one other variable that correlates with the variable to be measured. Therefore, the or one measured other variable or the combination of the measured other variables together are an indicator for the physical variable to be measured. The measurement provides at least one value for the physical variable sought.

The analyzer comprises its own output unit. On this output unit, the analyzer outputs the measured concentration of the substance in a visual form and/or in another form perceivable by a human. Optionally, the analyzer outputs an alarm on the output unit if the substance concentration lies outside a specified value range.

A valve in the interior of the main body can be moved into a closing end position and into a releasing end position. In the closing end position, the valve closes the fluid guide unit such that no fluid connection is established between the measurement chamber on the one hand and the suction opening in the input unit or the opening in the main body, and thus a surrounding environment of the analyzer, on the other hand. In the releasing (open) end position, the valve opens the fluid guide unit such that a gas sample can flow from the input unit through the fluid guide unit into the measurement chamber. Ideally, the valve being in the closing end position completely isolates the measurement chamber from the surrounding environment and from the input unit in a fluid-tight manner.

A filter is arranged in the fluid guide unit. The filter is located between the above-mentioned opening in the main body and the measurement chamber. If the input unit is connected to the main body, the filter is located between the suction opening and the valve. The filter can filter particles out of a gas sample flowing through the fluid guide unit and thus through the filter toward the measurement chamber. The wording that particles are filtered out means that the gas sample contains or may contain particles upstream of the filter, but thanks to the filter ideally no longer contains any particles downstream of the filter. In particular, the filter may act mechanically and/or may be electrostatically charged and thus attract and bind particles.

The filter usually comprises the actual filter element and a holder for the filter element. The gas sample flows through the filter element. The wording that the filter acts mechanically and/or is electrostatically charged means that the filter element has these properties.

According to the disclosure, a gas mixture to be analyzed can be introduced into the input unit through the inlet, and the introduced gas mixture flows through the input unit toward the outlet. The portion of the gas mixture that is not branched off as a gas sample and not sucked into the measurement chamber flows through the outlet into the surrounding environment. This feature is particularly advantageous if a test subject introduces the gas mixture into the input unit. This reduces the risk that the gas mixture introduced by the test subject flows into the face of a person holding the analyzer, wherein this person may be the actual test subject or another person holding the analyzer. Instead, the input unit can be positioned such that the gas mixture flows in a desired direction. It is not necessary to provide a retaining element in the input unit and/or to change the direction of flow of the gas mixture through the input unit.

The suction unit draws off the gas sample, by suction, from the gas mixture flowing through the input unit. This suction can be time-controlled, in particular by suitably actuating the suction unit. As a result, it is possible to draw off a by suction desired portion from the gas mixture by suction. In particular, it is possible to draw off, by suction, a specific amount and/or a specific proportion of the gas mixture, namely relatively independently of the volume of the gas mixture introduced into the input unit or the pressure or volume flow at which this gas mixture was or is introduced. In addition, it is possible to carry out the suction during a certain time period while the gas mixture is being introduced into the input unit.

If the introduced gas mixture is a breath sample from a test subject, in many cases the valve makes it possible that the gas sample drawn off by suction comprises exhaled air from at least one region of the respiratory system of the test subject, but does not comprise exhaled air from at least one other region of the respiratory system. By way of example, it is possible that the gas sample comprises exhaled air from the upper respiratory tract and/or the lungs, but not from the mouth area. This feature makes it easier to reliably measure the breath alcohol content in the breath sample, and thus in particular to determine whether or not there is alcohol in the test subject's blood.

According to the disclosure, in the closing end position, the valve can close the fluid guide unit in a fluid-tight manner and thus isolate the measurement chamber from the surrounding environment in a fluid-tight manner. On the one hand, this reduces the risk of evaporation of a component of a sensor. This risk exists particularly if the sensor is an electrochemical sensor. Without a valve, the risk of evaporation would occur particularly if the analyzer is stored for a relatively long time period, and therefore the measurement chamber is continuously fluidically connected to the surrounding environment via the fluid guide unit for a relatively long time period. On the other hand, the closed valve reduces the risk of particles and interfering substances, particularly interfering gases, entering the measurement chamber from outside through the fluid guide unit. Such particles and substances can cause the analyzer to malfunction.

In conjunction with a removable input unit, the use of the valve achieves the following advantage in particular: The valve eliminates the need to close the fluid guide unit by means of a cap. Such a cap would have to be removed before the input unit is connected to the main body. If the input unit is permanently connected to the main body, the cap would thus have to be placed both on the inlet and on the outlet and would have to be removed prior to use. This is time-consuming. The process of removing the cap usually requires action by a user.

According to the disclosure, a filter is located in the fluid guide unit, namely between the suction opening and the valve. The filter at this location has the following effect, particularly if the valve is in the releasing end position or in an open intermediate position and a gas sample is being sucked in through the fluid guide unit: The gas sample that is sucked in flows first through the filter and then through the valve-more precisely: through a filter element of the filter. Ideally, the filter filters out of the gas sample all particles that have a particular property, for example are larger than a specified upper limit, in practice at least a large proportion of these particles. The design of the filter determines which particles are filtered out. In the case of an electrostatically charged filter, the filter collects particles that have this specified property, for example all particles that are sufficiently large. “All particles” is an ideal scenario, which in reality can usually only be achieved approximately.

The filtering-out reduces the risk of the following undesirable event occurring: Particles flow with the gas sample to the valve and settle on a valve body and/or on a valve body seat or some other component of the valve or flow through the valve and enter the measurement chamber. Particles on the valve body or on the valve body seat may lead to the valve not closing the fluid guide unit in an entirely fluid-tight manner, even in the closing end position. This would lead to the risk that the measurement chamber is continuously fluidically connected to the surrounding environment, even if the valve is in the closing end position. Particles in the measurement chamber may lead to incorrect analysis results.

According to the disclosure, the filter is located in the fluid connection between the suction opening and the measurement chamber. This feature makes it possible to achieve the advantages of the filter, without it being necessary for a filter to be present in the input unit. The entire gas mixture introduced flows through the input unit, while only the branched-off portion of the gas mixture flows through the fluid guide unit. Therefore, more gas flows through the input unit than through the fluid guide unit, at least twice as much, particularly at least five times as much, in particular at least ten times as much. A filter in the input unit would therefore be more strongly charged by particles, and also mechanically loaded, than the filter according to the disclosure in the fluid guide unit. In addition, a filter in the input unit inevitably creates pneumatic resistance to a gas mixture flowing through, and the gas mixture would have to be introduced at a higher pressure. By way of example, a test subject would have to blow harder into the input unit. In addition, a filter in the input unit would in many cases increase the pneumatic resistance of the input unit compared to an input unit without a filter, which higher resistance could lead to undesirable back-pressure of the gas mixture in the input unit. This in turn could cause some of the gas mixture to flow into the test subject's face. This is undesirable.

In one embodiment, the filter is electrostatically charged and thereby separates out particles that flow through the fluid guide unit with the sucked-in gas sample. The particles flow past at least one charged electrode, or a plurality of charged electrodes, and are thereby ionized. An electric field causes the ionized particles to be attracted to an oppositely charged collecting electrode and to accumulate at the collecting electrode. In one implementation of an electrostatically charged filter, the filter comprises a nonwoven fabric. The gas sample flows through the nonwoven fabric. With particular preference, the nonwoven fabric used is a melt-blown nonwoven fabric.

In one embodiment, the filter acts mechanically. The filter can filter out particles from a gas sample, ideally all particles, if these particles are larger than an upper limit predefined by the design of the filter. In one implementation, the mechanical filter functions in the manner of a screen (surface filter).

In a different implementation, the filter comprises a plurality of (at least two, in one embodiment at least three) layers and/or is constructed as a porous body. Each layer comprises a plurality of fibers, and the arrangement of the fibers determines the position and size of each pore. It is not necessary for the pores to form a regular pattern. The fibers are arranged such that a single pore only allows a particle to pass through if the particle size is smaller than a predefined upper limit. The openings in different layers, i.e. the pores, are arranged offset from one another as follows: A particle that flows through the filter as part of the gas sample cannot pass through the filter in a straight line, but instead only if the particle changes its direction, i.e. is deflected (redirected), at least once, or a plurality of times. In the event of such a change in direction, the particle often sticks to the filter. In the case of such a depth filter, the openings (pores) may be larger than in the case of a screen (surface filter), without larger particles being able to pass through the filter.

In many cases, a depth filter has a lower pneumatic resistance than a surface filter, i.e. a filter that functions in the manner of a screen or sieve, while achieving the same filtering effect. In addition, a depth filter can often bind significantly more particles than a surface filter until the pneumatic resistance becomes so great that the filter has to be replaced. This feature means that the depth filter can often be used for longer than a surface filter.

The two embodiments, namely electrostatically charged filters and mechanical filters, can be combined with each other. Thanks to this combination, an electrostatically charged filter continues to filter out particles even if the electrostatic filter is discharged, which often happens after a relatively long period of use.

Particularly for the following reason, it is advantageous if the filter has a relatively low pneumatic resistance, which in particular is achieved by a filter that is configured as a depth filter and/or is electrostatically charged: With an otherwise constant suction process, the gas sample is sucked into the measurement chamber more quickly if there is a low pneumatic resistance compared with a high pneumatic resistance. A high speed is advantageous, for example, if the gas mixture introduced is a breath sample from a test subject: A high speed increases the reliability that the gas sample actually comes from a specific desired part of the test subject's respiratory system, for example from the lungs or the upper respiratory tract.

In one embodiment, the filter filters out, from the gas sample flowing through, all particles which have a maximum diameter that is smaller than or at most equal to an upper limit. This upper limit can be between 1 μm and 10 μm, particularly between 1 μm and 3 μm, and in particular is 2 μm.

The entire input unit or at least a mouthpiece can be detachably connected to the main body and can also be separated again from the main body. This embodiment makes it easier to comply with hygiene requirements. By way of example, a test subject introduces a breath sample into the input unit, and the input unit or at least the mouthpiece is used once for this one breath sample and then is discarded. However, the same main body can be used to analyze multiple gas samples one after the other. According to the disclosure, the filter is located in the interior of the main body and not in the input unit. Therefore, the filter can also be reused multiple times. Compared to a filter in the input unit, this reduces material consumption. In addition, the filter protects the valve from contamination from outside, even if the main body, i.e. the analyzer, is stored without an input unit. Thanks to the filter, it is less often necessary to place a cap onto an opening in the main body, with the fluid guide unit connecting this opening to the measurement chamber.

According to the disclosure, the suction unit draws off a gas sample, by suction, from the gas mixture flowing through the input unit and conveys it into the measurement chamber. The gas sample has a volume of less than 10% of the volume of the gas mixture flowing through the input unit, particularly less than 1%, in particular less than 0.1%. As a result, the filter is subjected to very little load and very few particles settle thereon, compared to the situation that a larger gas sample is drawn off.

Preferably, the valve comprises a valve body and a valve body seat. If the valve is in the closing end position, the valve body bears against the valve body seat, ideally in a fluid-tight manner. If the valve is in the releasing end position, a gap occurs between the valve body and the valve body seat.

In one embodiment, the analyzer comprises a mechanical connecting element. The connecting element mechanically connects the valve body of the valve to the suction unit. While the suction unit sucks in a gas sample and conveys it into the measurement chamber, the mechanical connecting element moves the valve from one end position to the other end position, preferably from the releasing end position to the closing end position. During this movement, the valve body is moved relative to the valve body seat. The suction unit moves the valve from the closing end position to the releasing end position prior to sucking in a gas sample and moves it back from the releasing end position to the closing end position during the suction process. The two processes of sucking in the gas sample and opening the valve are therefore synchronized with each other and temporally overlap. The fluid guide unit is then closed again after the suction process. The inverse implementation is also possible. The connecting element in the main body can execute two linear movements in two opposite directions.

Thanks to the mechanical connecting element, it is sufficient that the analyzer comprises a single drive. On the one hand, this single drive can move the suction unit and thus convey a gas sample into the measurement chamber. On the other hand, this drive can move the valve from one end position to the other end position. Thanks to the connecting element, it is not necessary to provide two different drives. This saves installation space.

This embodiment enables the following mode of operation: As long as no gas sample is being sucked in, the valve is in the closing end position. In order to suck in a gas sample, the following two steps are carried out one after the other: In the first step, the suction unit conveys gas from a reservoir into the measurement chamber and thus flushes the measurement chamber. The reservoir is located in the interior of the main body. During the flushing-out process, gas is conveyed from the measurement chamber through the fluid guide unit out of the housing of the analyzer and into the input unit. At the same time, or at least in a temporally overlapping manner, the valve body is moved away from the valve body seat by means of the connecting element, and the valve is moved to the releasing end position. The gas from the measurement chamber is conveyed out of the analyzer. In the second step, the suction unit sucks in a gas sample from the input unit. The gas sample flows through the fluid guide unit into the measurement chamber. At the same time, or at least in a temporally overlapping manner, the valve body is moved back onto the valve body seat by means of the connecting element, and the valve is moved back to the closing end position. As a result, the valve closes the fluid guide unit for as long as possible, and the measurement chamber is only fluidically connected to the surrounding environment for as long as necessary. The closed valve and the filter isolate the measurement chamber from the surrounding environment even if the input unit is separated from the main body.

In one embodiment, the suction unit comprises a chamber with a variable volume, in particular a bellows or a piston-cylinder unit. The measurement chamber is located between the chamber of the suction unit and the fluid guide unit. The chamber is fluidically connected to the measurement chamber. The suction unit chamber provides the reservoir described above. While the volume of the chamber is increased, a negative pressure is thus generated in the chamber of the suction unit, hence in the measurement chamber and hence in the fluid guide unit, and a gas sample is sucked through the fluid guide unit into the measurement chamber as a result of the negative pressure. While the volume of the chamber is reduced, a positive pressure is generated in the chamber of the suction unit and hence in the measurement chamber, and the measurement chamber is flushed out as a result.

The mechanical connecting element connects the suction unit to the valve as follows: If the chamber is moved into the minimum-volume state, the valve is moved to the releasing end position. Conversely, if the chamber is moved into the maximum-volume state, the valve is moved to the closing end position.

The analyzer comprises a heater. The heater can heat a segment of the fluid guide unit. The filter is located in the heatable segment. The heater can comprise a wire, which is electrically conductive and heats up while current flows through it. In a different implementation, the heater heats the segment in a contactless manner. A radiation source emits electromagnetic radiation, in particular infrared radiation, and the emitted radiation heats the segment.

In many cases, the gas mixture flowing through the input unit includes liquid droplets and/or vapor. This applies particularly if the gas mixture is a breath sample emitted by a test subject. The gas sample drawn off by suction therefore usually also includes liquid droplets and/or vapor. There is a risk that liquid will condense on a filter element of the filter in the fluid guide unit and, for example, drip off the filter. This condensed liquid could lead to a greater pneumatic resistance and/or could damage the filter element and/or could falsify a measurement result of the sensor arrangement. In particular, liquid could condense on an inner wall of the fluid guide unit or on an optical element of the sensor arrangement.

As is known, under otherwise constant conditions, the higher the temperature of a gas mixture is, the more liquid the gas mixture can absorb. The heater therefore reduces the risk of liquid condensing on the filter. Because the filter is located in the heated segment, the risk of condensation is lower than if the heater were located elsewhere.

The tubular input unit extends along a longitudinal axis. The fluid guide unit likewise extends along a longitudinal axis. These two longitudinal axes enclose an angle of at least 60 degrees between them. With particular preference, the two longitudinal axes are perpendicular to each other. This embodiment makes it easier for the longitudinal axis of the input unit to be arranged approximately horizontally and for the longitudinal axis of the fluid guide unit to be arranged approximately vertically while the analyzer is in use. The main body can be comfortably held in one hand. The direction in which the gas mixture leaves the input unit can easily be defined.

In one embodiment, the analyzer comprises a pressure sensor. The pressure sensor can measure the pressure at a first measurement position at least once, also repeatedly, in particular at a fixed sampling frequency. The first measurement position is located downstream of the filter and outside of the input unit. In a first implementation, the first measurement position is located in or on the fluid guide unit between the filter and the measurement chamber. In a second implementation, it is in the measurement chamber or on a wall of the measurement chamber, and in a third implementation between the measurement chamber and the suction unit. The first measurement position is therefore located in the interior of the main body and outside the input unit, and therefore the pressure at the first measurement position often depends relatively little on the pressure at which a gas mixture has been introduced into the input unit.

The analyzer can determine the pressure at the first measurement position, including the temporal course of this pressure, and can use at least one measured value, including a signal, i.e. a sequence of measured values, from the pressure sensor for this purpose. The pressure at the first measurement position matches-usually after a transient phase-the pressure in the measurement chamber, usually both if the valve is open and if the valve is closed. For as long as the valve is closed, the pressure at the first measurement position may differ from the ambient pressure and from the pressure in the input unit.

Optionally, the pressure sensor can additionally measure the pressure at a second measurement position. The second measurement position is likewise located in or on the fluid guide unit, but upstream of the filter. If the input unit is attached, the second measurement position is therefore located between the suction opening and the filter. The analyzer can determine the pressure, including the temporal course of the pressure, at the second measurement position and can use at least one further measured value, including the signal, from the pressure sensor for this purpose.

Therefore, as seen in the direction of flow of a gas sample from the input unit into the measurement chamber, the first measurement position is located downstream of the filter and the optional second measurement position is located upstream of the filter. In other words: The filter is located between the two measurement positions.

The terms “upstream” and “downstream” relate to the flow direction of a gas sample from the input unit into the measurement chamber.

The analyzer can determine a current pneumatic resistance of the filter. The pneumatic resistance of the filter is the quotient of the pressure difference (numerator) and the volume flow through the filter (denominator), with the pressure difference being the pressure difference between the pressure upstream and the pressure downstream of the filter, i.e. the pressure drop across the filter. In many cases, it is justified to assume that the pneumatic resistance does not significantly depend on the volume flow and therefore the pressure difference can be assumed to be proportional to the volume flow.

Embodiments of how the analyzer can automatically determine the pneumatic resistance of the filter will be described below.

The analyzer—more precisely: a signal-processing evaluation unit of the analyzer, for example a control unit—can determine (measure or capture) a volume flow through the filter and thus through the fluid guide unit. This volume flow occurs if the input unit is attached, the valve is opened, and the suction unit is activated.

Different implementations as to how the analyzer can measure or otherwise determine the volume flow are possible. It is also possible that the evaluation unit determines a mass flow instead of or in addition to the volume flow. The volume flow is the volume per time unit that flows through a fluid guide unit; the mass flow is the mass per time unit.

In a first implementation, the analyzer determines the volume flow as a function of an actuation and/or a property of the suction unit. This suction unit causes the volume flow by suction. Usually, the geometry of the suction unit determines the volume of a gas sample sucked in. The duration of a suction process results from the actuation. The quotient gives at least approximately the volume flow sought. In a different implementation, the analyzer comprises a volume flow sensor.

In a second implementation, the analyzer derives the volume flow using the measured pressure at the first measurement position and the measured pressure at the second measurement position. Based on the measured values from the pressure sensor, the analyzer derives a temporal course of the difference between the pressures at the two measurement positions. This pressure difference is the pressure drop across the filter. From the temporal course of the pressure difference, the analyzer derives the volume flow. If the pneumatic resistance of the filter has been measured in another way, the analyzer derives the volume flow from the pressure difference and the pneumatic resistance.

In a third implementation, the analyzer is configured to approximately determine the volume flow through the filter by the following steps:
The pressure sensor measures the pressure at the first measurement position repeatedly.
The analyzer determines a time period during which a sufficiently large negative pressure relative to a reference pressure occurs at the first measurement position. To determine this time period, the analyzer uses the temporal course of the pressure at the first measurement position.
The reference pressure is, for example, the pressure measured by the pressure sensor at the first measurement position at at least one time point at which the valve was closed and therefore no gas sample was being sucked in. The reference pressure can also be a pressure measured in a surrounding environment of the analyzer (ambient pressure).
The negative pressure causes the gas sample to be sucked in. From the determined time period, the analyzer derives the time taken to suck in the gas sample.
The analyzer determines the volume of the gas sample sucked in. The volume of the gas sample sucked in is determined with sufficient accuracy through the geometry and/or design of the suction unit, in particular through the volume of a chamber of the suction unit, and the geometry and design are known in advance. Usually, the volume of the gas sample does not significantly depend on the volume of the gas mixture introduced into the input unit.
The suction unit uses as the volume flow the quotient of the volume of the gas sample and the determined time taken.

The third implementation does not require that the pressure is measured at a second measurement position upstream of the filter or that the duration of a suction process is derived from the actuation of the suction unit. Furthermore, the third implementation does not require that the pneumatic resistance of the filter is known. Instead, the pneumatic resistance can be derived by means of the third implementation.

According to a fourth implementation, the analyzer additionally comprises a volume flow sensor, wherein the volume flow sensor measures the volume flow through the filter and uses the principle of thermal anemometry or the principle of laser Doppler anemometry for this purpose. The fourth implementation also does not require that the pressure is measured at the second measurement position or that the pneumatic resistance of the filter is known. Instead, according to the fourth implementation, too, the pneumatic resistance can be derived.

One application of the embodiments just described, by which the volume flow is determined, will be set out below. The analyzer—more precisely: a signal-processing evaluation unit of the analyzer, for example a control unit—can determine the current pneumatic resistance of the filter. For the application described below, it will be assumed that the pneumatic resistance does not depend on the volume flow and therefore the pressure drop across the filter is proportional to the volume flow.

Implementations for determining the volume flow, without knowing the pneumatic resistance of the filter, have been described above. Different implementations are possible as to how the analyzer measures the pressure drop across the filter.

In one implementation, the pressure sensor measures the pressure at the first measurement position and the pressure at the second measurement position, with the first measurement position being arranged downstream of the filter and the second measurement position being arranged upstream of the filter. The difference between the two measured pressures gives the pressure drop.

In another embodiment, the analyzer uses the time period mentioned above, wherein during this time period a negative pressure relative to a reference pressure occurs at the first measurement position. To derive the pressure drop from this determined time period, the following assumption is used: If no filter were arranged in the fluid guide unit, the following sequence would occur:
The suction unit sucks in the gas sample.
The valve is opened.
Once the valve has been opened, a transient phase occurs, during which pressure differences along the fluid guide unit disappear. After this transient phase, the pressure in the measurement chamber matches the pressure in the fluid guide unit, namely upstream of the filter. In particular, the pressure in the measurement chamber matches the pressure at the first measurement position.

After the transient phase, a difference between the pressure at the suction opening and the pressure at the first measurement position is substantially defined by the pneumatic resistance of the filter. Therefore, the analyzer uses as the pressure drop a time-averaged or maximum difference between the pressure at the first measurement position and the reference pressure mentioned above. This time period occurs during the time interval in which the gas sample is being sucked in, and it occurs after the transient phase.

Different implementations as to how the analyzer measures or otherwise determines the volume flow through the filter, without using the pneumatic resistance, have been described above. The analyzer determines the current pneumatic resistance of the filter as the quotient of the pressure drop across the filter and the volume flow through the filter.

Due to the fact that the filter filters particles out of the gas sample flowing through it, usually the pneumatic resistance of the filter increases. The control unit determines the current pneumatic resistance repeatedly, for example whenever N gas samples have been sucked in since the last determination of the pneumatic resistance, where N>=1 is a predefined number, or if the gas samples that have flowed through the filter since the last determination have a total volume and/or mass above a specified upper limit.

The analyzer can be configured as follows: If the pneumatic resistance lies outside a specified values range, the analyzer generates a message to this effect. The value range is characterized at least by an upper limit, and optionally additionally by a lower limit that is greater than zero. If the pneumatic resistance is above the upper limit, the message includes the indication that the filter needs to be replaced. The analyzer outputs this message in at least one form perceivable by a human, such as on an output unit of the analyzer itself. This message is therefore directed to a user of the analyzer. It is also possible that this message is additionally or instead transmitted to a spatially remote receiver and is output on an output unit of the receiver.

In one implementation, the analyzer compares the pneumatic resistance not only with the specified upper limit, but also with a specified lower limit. This lower limit is specified, for example, as follows: If the filter is correctly inserted and the analyzer has not yet been used, i.e. no particles have yet settled on the filter, the pneumatic resistance of the filter is equal to or greater than the lower limit. Therefore, if the measured pneumatic resistance is lower than the lower limit, an error has occurred. Possible causes for this error are, in particular, the following:
No filter is inserted in the fluid guide unit.
The filter is not inserted correctly, so that some of the gas sample drawn off by suction can bypass the filter.
The filter is damaged, for example it has a crack, so that some of the gas sample can flow through the filter without the filter filtering out particles.

A message is generated and output also if the pneumatic resistance is too low.

In one embodiment, the analyzer can automatically check whether a filter is or is not inserted in the main body, for example by means of a contact switch or by the analyzer determining the pneumatic resistance in the fluid guide unit. In another embodiment, the analyzer can detect a confirmation by a user that a filter has been inserted. It is possible that the suction unit is only activated and only sucks in a gas sample if a filter is inserted and the pneumatic resistance lies in the specified value range.

A filter with a high pneumatic resistance can lead to the situation where a gas sample is sucked into the measurement chamber too slowly and/or with a too small volume, and as a result an incorrect, in particular too low, concentration of the substance is measured. Other undesired effects are as follows:
A higher kinetic energy is required for suction.
Introduction of the gas mixture takes a relatively long time.

A high required kinetic energy can result in a relatively large amount of electrical energy being consumed. This is particularly disadvantageous while the analyzer is not connected to a stationary power supply network and therefore is supplied by its own power supply unit. As already explained, the following disadvantages occur in particular if a gas sample is being sucked in relatively slowly from an introduced breath sample: The gas sample then often no longer comes only from a desired part of the test subject's respiratory system, for example from the upper respiratory tract or the lungs, but also from another part.

The embodiment in which the pneumatic resistance of the filter is measured enables the analyzer to automatically check whether the filter can further be used or whether it has become so heavily clogged with filtered-out particles that the filter needs to be replaced. It is not necessary to replace the filter purely based on time or solely as a function of the number of gas samples drawn off by suction, i.e. regardless of the current condition of the filter. It is possible, but thanks to the embodiment not absolutely necessary, for a person to visually check the filter to ascertain its current condition. The embodiment just described, in which the pneumatic resistance of the filter is measured, is particularly advantageous over a visual inspection if the filtered-out particles visually differ only slightly from a filter element of the filter. In addition, it is not necessary to weigh the filter to determine its current condition.

The filter is configured as an electrostatically charged and/or mechanically acting element. In both embodiments, the filter has no chemical or thermal influence on a gas sample flowing through the filter, ideally no influence at all. Therefore, the filter also does not falsify a measurement result of the analyzer.

According to the disclosure, the sensor can measure the concentration of the substance in a gas sample located in the measurement chamber. Different operating principles for this sensor are possible.

For example, the substance is a combustible gas, and the sensor is an electrochemical sensor and works on the principle of a fuel cell. The electrical charge generated is an indicator for the content of the combustible gas in the gas sample. The sensor can also be a photoelectric sensor. Electromagnetic radiation penetrates the measurement chamber, and the substance to be detected attenuates electromagnetic radiation in a specific wavelength range. Therefore, the measured attenuation correlates with the content of the substance. The sensor can also be a photoacoustic sensor. Electromagnetic radiation causes an acoustic effect, and the substance modifies this acoustic effect. In the case of a photoionization detector, the electromagnetic radiation ionizes molecules. It is also possible that the sensor is equipped as a so-called heat-tone sensor, wherein a detector oxidizes a combustible substance, the oxidation of the combustible substance releases heat energy, and the released heat energy is measured and is an indicator for the concentration of the combustible substance.

In one implementation, the analyzer comprises two sensors, which are connected in parallel or in series with respect to the gas sample. These two sensors can use the same or different measuring principles. The embodiment with two differently operating sensors creates redundancy and, in many cases, increases the reliability that the concentration and/or amount of the substance will be correctly measured.

The disclosure further relates to a monitoring unit which can monitor an analyzer according to the disclosure. The monitoring unit comprises the pressure sensor described above, as well as a signal-processing evaluation unit. The evaluation unit can be a component of a control unit of the analyzer or else can be arranged spatially separate (remote) from the analyzer. The pressure sensor can measure the pressure at the first measurement position. The evaluation unit can receive a signal of the pressure sensor and can determine the current pneumatic resistance of the filter and compare it with the specified value range. If the pneumatic resistance lies outside the value range, the evaluation unit generates a message and causes the latter to be output in a form perceivable by a human.

### BRIEF DESCRIPTION OF DRAWINGS

The disclosure will be described below on the basis of an exemplary embodiment. In the drawings,

FIG. 1 schematically shows the mode of operation of an electrochemical sensor;

FIG. 2 shows, in a perspective view obliquely from above, a first embodiment of the analyzer according to the disclosure;

FIG. 3 shows, in a view vertically from above, the analyzer of FIG. 2;

FIG. 4 shows, in a cross-sectional view, the analyzer of FIG. 2, wherein the input unit is omitted;

FIG. 5 shows, in a perspective view almost vertically from above, a second embodiment of the analyzer according to the disclosure;

FIG. 6 shows, in a cross-sectional view, the analyzer of FIG. 5;

FIG. 7 shows, in a perspective view almost vertically from above, a third embodiment of the analyzer according to the disclosure, wherein the input unit is omitted;

FIG. 8 shows, in a further cross-sectional view, the analyzer of FIG. 7, wherein the input unit is omitted.

### DESCRIPTION

The analyzer according to the disclosure can analyze a gas mixture for a specified substance. In the exemplary embodiment, the gas mixture is a breath sample exhaled by a test subject to be analyzed. In the exemplary embodiment, the substance is breath alcohol. The task is to analyze the test subject to determine whether or not his/her blood contains alcohol above a detection limit. If the test subject has consumed alcohol and the alcohol in the blood has not yet fully broken down, it is known that the provided breath sample will contain breath alcohol. The disclosure can also be used for a different substance that can be detected in a breath sample from a test subject.

The test subject introduces a breath sample into an input unit. A portion of the breath sample is drawn off from the input unit by suction and flows into a measurement chamber. A sensor in or on the measurement chamber measures the breath alcohol content in the gas sample. More precisely: The sensor measures a physical detection variable that correlates with the content (concentration) of breath alcohol in the gas sample located in the measurement chamber and is therefore an indicator for the alcohol content.

The sensor generates a signal. The generated signal includes information about the measured breath alcohol content. For example, the sensor measures the amount of breath alcohol in the gas sample, and a signal-processing evaluation unit derives the concentration of breath alcohol in the gas sample and thus in the breath sample from the amount of breath alcohol and the volume of the measurement chamber.

In the exemplary embodiment, the analyzer is a device which a person can hold in his/her hand and hold in front of the test subject's face. The analyzer comprises its own power supply unit and its own output unit. The test subject introduces the breath sample into the input unit. The measured breath alcohol content is displayed on the output unit in at least one form perceivable by a human. Optionally, it is also indicated whether the measured breath alcohol content is above a specified limit. This limit is specified, for example, by legal regulations for car drivers and other vehicle or plant operators.

Different principles have become known from the prior art as to how a sensor can measure the concentration of a substance in a gas mixture. A number of these principles can also be used for the disclosure. The sensor of the analyzer according to the exemplary embodiment is, for example, an electrochemical sensor, a photo optical sensor, a photoacoustic sensor, a photoionization sensor, or a heat-tone sensor (catalytic sensor, pellistor sensor).

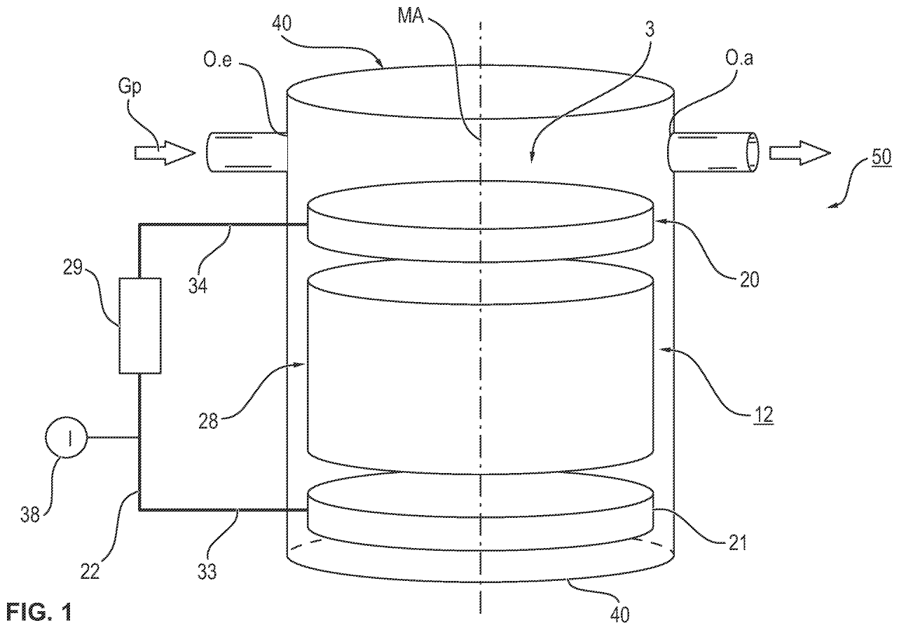

In one implementation, the analyzer comprises an electrochemical sensor. FIG. 1 schematically shows, by way of example, the mode of operation of an electrochemical sensor 12, this mode of operation being known from the prior art. The sensor 12 operates in accordance with the fuel cell principle, with breath alcohol as the fuel. Breath alcohol creates a chemical reaction in the measurement chamber. This chemical reaction triggers the step that electrical current flows. The electrical charge is measured and is an indicator for the breath alcohol content in the gas sample Gp located in a measurement chamber 3.

The drawing in FIG. 1 is not necessarily true to scale. Reference sign 50 denotes a sensor arrangement. The sensor arrangement 50 comprises the sensor 12 and the measurement chamber 3, which is surrounded by a wall 40. In the exemplary embodiment, the measurement chamber 3 has the shape of a cylinder, which is rotationally symmetrical relative to a central axis MA. Of course, other geometric shapes are also possible.

A gas sample Gp flows into the measurement chamber 3 through an inlet O.e and flows back out of the measurement chamber 3 through an outlet O.a. It is also possible that the gas sample Gp flows back out of the measurement chamber 3 through the inlet O.e.

The electrochemical sensor 12 comprises
a measuring electrode 20, which is electrically contacted by a contact wire 34,
a counter-electrode 21, which is electrically contacted by a contact wire 33,
an electrolyte 28 between the two electrodes 20 and 21,
a connecting wire 22, which electrically connects the two contact wires 33 and 34 to each other,
an electrical measuring resistor 29 in the connecting wire 22, and
a current intensity sensor 38, which measures the intensity I of the current flowing through the connecting wire 22.

The electrolyte 28 comprises an electrolytically conductive medium, for example sulfuric acid or phosphoric acid or perchloric acid diluted with water. In one implementation, a porous membrane provides the electrolyte 28. Ions can move in the electrolyte 28. The electrolyte 28 establishes an ionically conductive connection between the measuring electrode 20 and the counter-electrode 21, but prevents electrons from flowing between the two electrodes 20 and 21. The gas sample Gp reaches the measuring electrode 20, but not the counter-electrode 21. The two contact wires 33 and 34 are electrically conductive and are made of a material that is not chemically attacked by the electrolyte 28, for example are made of platinum or gold. The electrodes 20 and 21 are also made of a chemically resistant material, for example are likewise made of platinum or gold.

As already explained, the substance to be detected, in this case breath alcohol, triggers a chemical reaction, during which the substance to be detected is oxidized-of course only if a sufficient amount of the substance is present in the gas sample Gp. As a result of the chemical reaction, an electric current flows between the measuring electrode 20 and the counter-electrode 21 and thus through the connecting wire 22. The current intensity sensor 38 measures the current intensity I. An evaluation unit derives the electrical charge Q, i.e. the total amount of electrical current flowing through the connecting wire 22 (principle of coulometry). Usually, the electric current flows until the entire amount of the substance to be detected, i.e. in this case all the breath alcohol or another oxidizable substance, is oxidized in the measurement chamber 3. The electrical charge correlates with the breath alcohol content in the gas sample Gp.

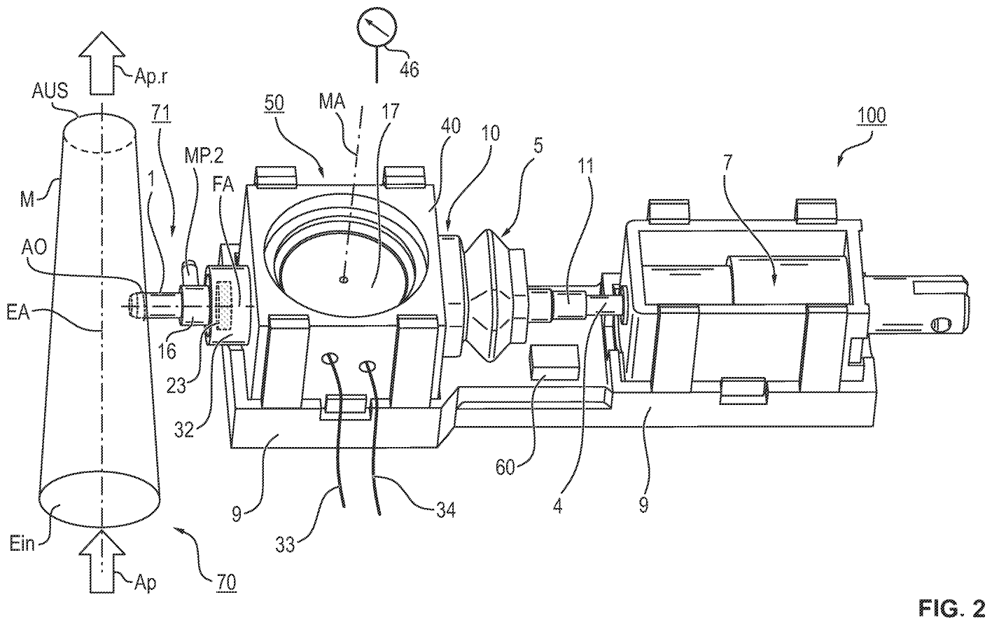

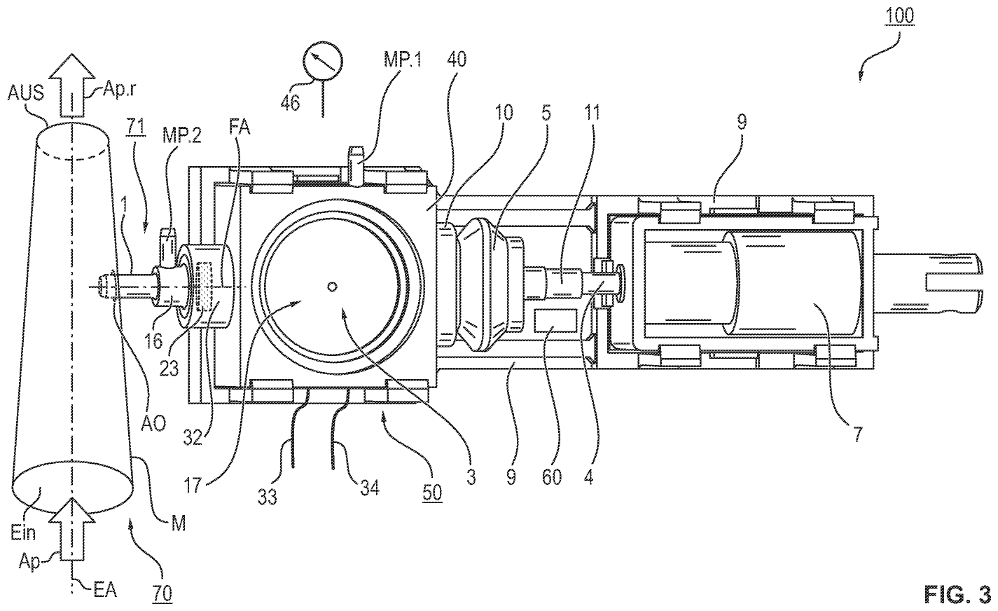

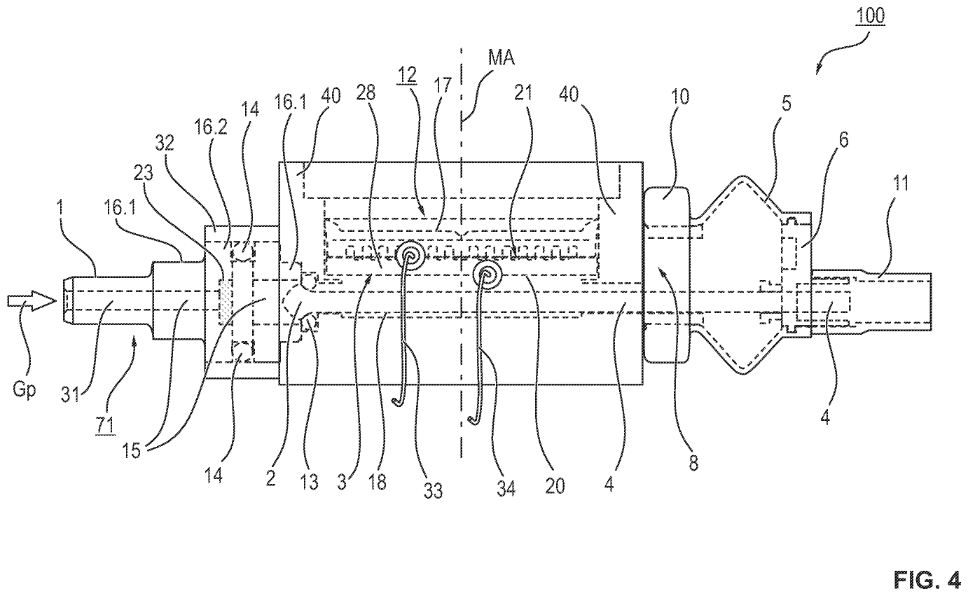

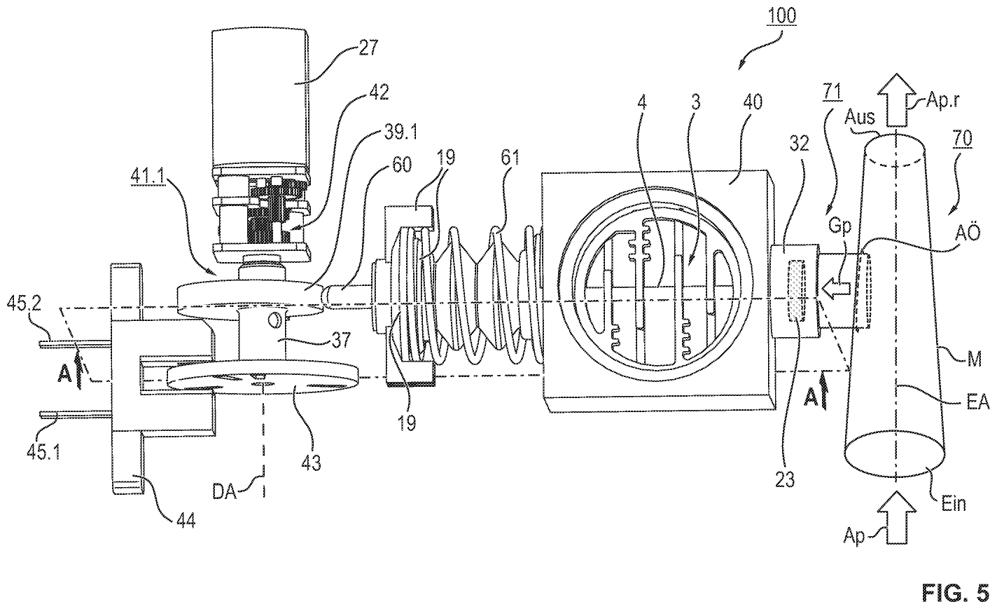

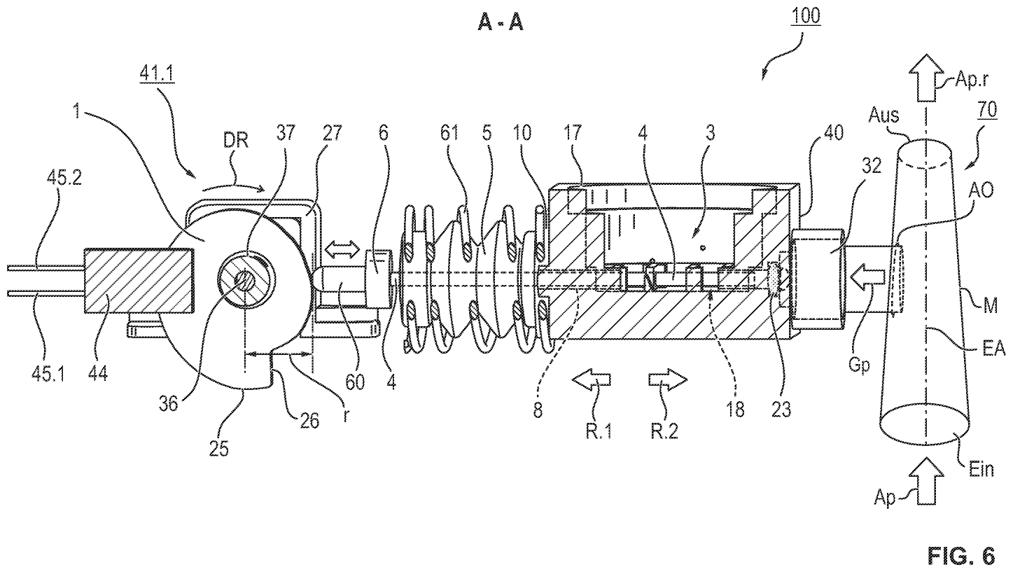

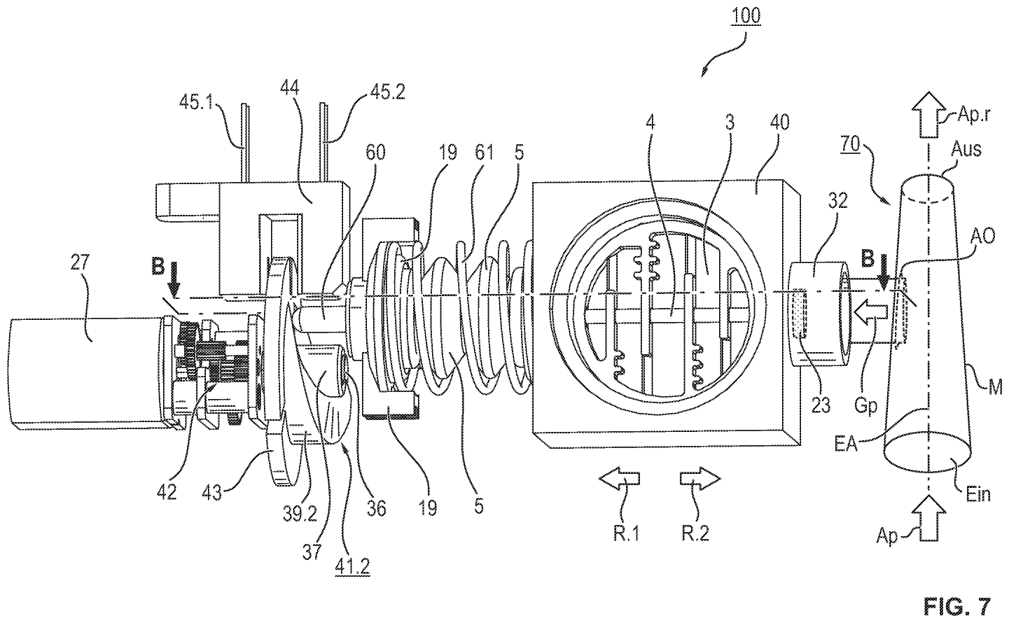

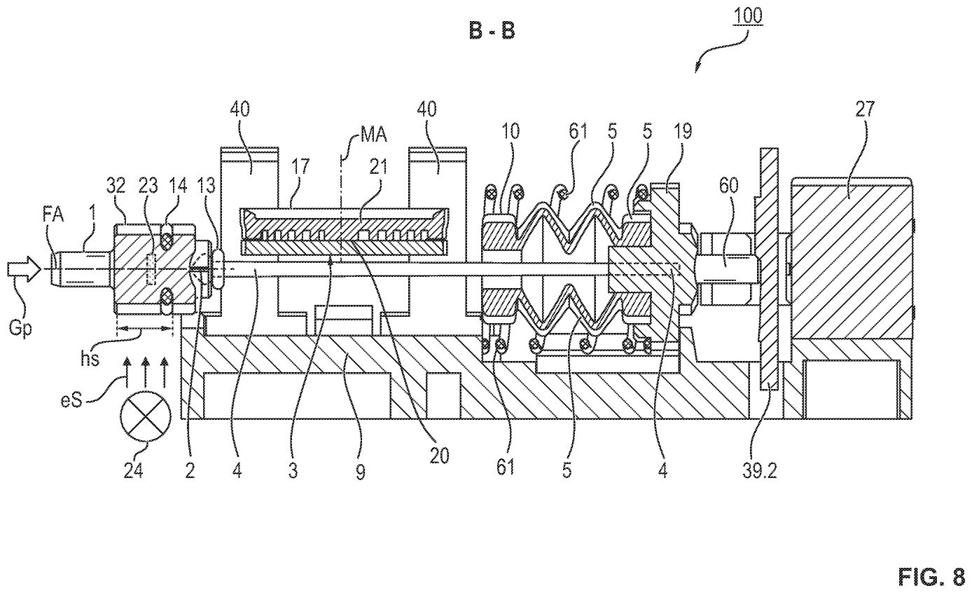

FIG. 2 to FIG. 4 show a first embodiment of the analyzer according to the disclosure, FIGS. 5 and 6 show a second embodiment, and FIGS. 7 and 8 show a third embodiment. The same reference signs have the same meanings and also the meanings of FIG. 1. FIG. 2, FIG. 3, FIG. 5 and FIG. 7 show the analyzer 100 in perspective views vertically or obliquely from above, FIG. 4 shows the first embodiment in a cross-sectional view from the side, and FIG. 6 and FIG. 8 show the second and the third embodiment, respectively, in two different cross-sectional views from the side.

In all embodiments, the analyzer 100 comprises a tubular input unit 70. The input unit 70 comprises
an inlet In,
an outlet Out,
a tubular lateral surface M between the inlet In and the outlet Out, and
a suction opening AO in the lateral surface M.

The input unit 70 is shown schematically in FIG. 2, FIG. 3, FIG. 5, FIG. 6 and FIG. 7 and is omitted in the other figures. In the exemplary embodiment, the input unit 70 has the shape of a funnel that tapers from the inlet In to the outlet Out. The input unit 70 has a central axis EA. In the exemplary embodiment, the input unit 70 is approximately rotationally symmetrical relative to the central axis EA.

While a test subject is to introduce a breath sample Ap, the analyzer 100 is held in front of the test subject's mouth such that the inlet In points toward the mouth. The test subject introduces the breath sample Ap into the input unit 70. The introduced breath sample Ap flows through the inlet In into the input unit 70 and parallel to the central axis EA through the input unit 70 to the outlet Out. A portion of the breath sample Ap is drawn off by suction through the suction opening AO, which will be described in greater detail below. The remainder Ap. r of the breath sample Ap that has not been branched off flows through the outlet Out into the surrounding environment. The portion of the breath sample Ap that has been drawn off by suction will later be fed back into the input unit 70.

A housing surrounds a main body of the analyzer 100. A user holds this main body in one hand while the test subject provides the breath sample Ap. Of the main body, only a frame 9 can be seen.

The sensor arrangement 50 comprising the measurement chamber 3 and the sensor 12 is mounted on the frame 9, wherein the sensor arrangement 50 can be configured as described with reference to FIG. 1. A wall 40 surrounds the cylindrical measurement chamber 3. The outer surface of the wall 40 has approximately the shape of a cuboid. A cover plate 17 is placed onto the wall 40.

A fluid guide unit 71 connects the suction opening AO in the input unit 70 to the measurement chamber 3. The fluid guide unit 71 extends along a longitudinal axis FA. In the exemplary embodiment, the longitudinal axis FA of the fluid guide unit 71 is perpendicular to the longitudinal axis EA of the connected input unit 70. In general, the longitudinal axis FA encloses an angle of at least 60° with the central axis MA.

The fluid guide unit 71 comprises
a hollow tip 1, which is connected to the input unit 70,
a hollow connecting piece 16 comprising a smaller part 16.1 and a larger part 16.2, and
an inflow-side connector 32.

The fluid guide unit 71 establishes a fluid connection between the input unit 70 and the measurement chamber 3. The fluid connection comprises a segment 31 in the hollow tip 1, a segment 15 in the hollow connecting piece 16, and a segment 18 beneath the measurement chamber 3. The segment 18 is fluidically connected to the measurement chamber 3.

In one embodiment, either the input unit 70 with the suction opening AO can be placed onto the tip 1, or a cap (not shown) can be placed onto the latter. According to this embodiment, the cap is placed on if the analyzer 100 is not in use and therefore if no input unit is in place. Before an input unit 70 is placed onto the tip 1, the cap must be removed. Otherwise, the input unit 70 usually cannot be placed on. Conversely, the input unit 70 must be removed in order to put the cap in place. It is possible to use such a cap, but thanks to a filter described below this is not necessary.

It is possible that the input unit 70 is used once and then is discarded. In this embodiment, the input unit 70 is detachably connected to the tip 1. This embodiment enables a test subject to place the input unit 70 in his/her mouth or to hold it close to his/her face.

The measurement chamber 3 in the wall 40 is located between the fluid guide unit 71 and a suction unit. The suction unit sucks a gas sample Gp out of the input unit 70, through the fluid guide unit 71, and into the measurement chamber 3. In the exemplary embodiment, the suction unit can also flush out the measurement chamber 3, with an “old” gas sample in the measurement chamber 3 being expelled through the fluid guide unit 71 and into the input unit 70. In one embodiment, the old gas sample is expelled from the measurement chamber 3 if an input unit 70 is connected to the tip 1; in another embodiment, it is expelled if the tip 1 is not connected to an input unit.

In the exemplary embodiment, the suction unit comprises a bellows 5 with a variable volume. The bellows 5 is fastened to a tubular, outflow-side connector 10. The connector 10 is fastened to the wall 40 and establishes a fluid connection 8 between the measurement chamber 3 and the interior of the bellows 5. The wall 40 is located between the two connectors 32 and 10.

The bellows 5 has a variable volume. While the volume of the bellows 5 is increased, a negative pressure occurs in the bellows 5, whereby gas is sucked in and a gas sample Gp is sucked through the fluid guide unit 71 into the measurement chamber 3. Conversely, while the volume of the bellows 5 is reduced, a positive pressure occurs, whereby gas is expelled from the bellows 5 and a gas sample Gp is expelled from the measurement chamber 3 and pushed into the fluid guide unit 71. The measurement chamber 3 is thus flushed out. The volume of the gas sample Gp sucked in is usually approximately equal to the difference between the largest and the smallest volume of the bellows 5.

Arranged in the interior of the bellows 5 is a plate 6, which is connected to a sleeve 11, see FIG. 4. The sleeve 11 is connected to a rod 4. A linear movement of the rod 4 parallel to the longitudinal axis FA away from the fluid guide unit 71 increases the volume of the bellows 5. A linear movement of the rod 4 in the opposite direction reduces the volume of the bellows 5.

A valve can selectively close or open the fluid guide unit 71. The valve comprises a valve body 2 and a valve body seat 13 in the form of a sealing ring. If the valve 2, 13 is closed, the valve body 2 bears against the valve body seat 13, ideally in a fluid-tight manner. If a gap occurs between the valve body 2 and the valve body seat 13, the valve 2, 13 opens the fluid connection 71. Only if the valve 2, 13 opens the fluid guide unit 71, a gas sample Gp can be sucked in from the input unit 70 and can flow through the fluid guide unit 71. FIG. 4 shows the valve 2, 13 in an open (releasing) state.

In the exemplary embodiment, the valve body 2 sits on the end of the rod 4 pointing toward the input unit 70. A movement of the rod 4 not only changes the volume of the bellows 5, but also moves the valve body 2 relative to the valve body seat 13. Thanks to the rod 4, the process of sucking in a gas sample Gp through the fluid guide unit 71 or flushing out the measurement chamber 3 is synchronized with the process of opening or closing the valve 2, 13. In particular, in one implementation, it is possible that the valve 2, 13 is only opened while a gas sample Gp is to be sucked in or the measurement chamber 3 is to be flushed out, and otherwise it is closed.

In one embodiment, the following sequence is carried out in order to introduce a new gas sample Gp into the measurement chamber 3:
Initially, the valve 2, 13 is closed. The bellows 5 has the maximum volume.
The rod 4 is moved toward the input unit 70. As a result, the volume of the bellows 5 is reduced, the measurement chamber 3 is flushed out, and the valve 2, 13 is opened. The gas previously located in the measurement chamber 3 is conveyed through the fluid guide unit 71 into the input unit 70.
The rod 4 is then moved away from the input unit 70 again. The volume of the bellows 5 is increased, and a gas sample Gp from the input unit 70 is sucked through the fluid guide unit 71 into the measurement chamber 3.
The sucking-in of the gas sample Gp is concluded, and the movement of the rod 4 is ended, if the valve body 2 has reached the valve body seat 13 and thus the valve 2, 13 is once again closed.

FIG. 4 also shows a sealing ring 14.

In the first embodiment, an actuating drive can move the rod 4 linearly in two anti-parallel directions R.1 and R.2. A return element (not shown) strives to move the rod 4 away from the input unit 70 and thereby close the valve 2, 13 and move the bellows 5 into a maximum-volume state and keep it there. A solenoid 7 can be activated by applying current to the solenoid 7, and it can be deactivated again. The activated solenoid 7 strives to move the rod 4 toward the input unit 70, namely counter to the force of the return element, and thereby strives to reduce the volume of the bellows 5 and open the valve 2, 13. Activating the solenoid 7 causes the measurement chamber 3 to be flushed out. As soon as the solenoid 7 is deactivated again, the return element moves the rod 4 away from the input unit 70, and the moved rod 4 moves the bellows 5 into the maximum-volume state. Thanks to the return element, the process of moving the valve 2, 13 to the closing end position and keeping it there does not consume any electrical energy.

In the first embodiment, an actuating drive comprising a solenoid 7 can move the rod 4 linearly back and forth. In the second and the third embodiment, the rod 4 is likewise moved linearly in two anti-parallel directions R.1 and R.2, but not by an actuating drive, but instead by a motor 27, such as an electric motor 27. The motor 27 rotates an output shaft 36 about an axis of rotation DA via a reduction gear 42, see FIG. 5. In the exemplary embodiment, the axis of rotation DA of the output shaft 36 is arranged parallel to the longitudinal axis EA of the input unit 70, but the parallel arrangement is not necessary.

Thanks to the implementation described below, it is sufficient that the motor 27 can be switched on and off and that the switched-on motor 27 can always rotate the output shaft 36 in the same direction of rotation DR about the axis of rotation DA. It is not necessary to provide an actuating drive that executes an oscillating movement.

A transmission unit 41.1 (second embodiment) or 41.2 (third embodiment) generates an oscillating movement of the rod 4 from the continuous rotation of the output shaft 36. The transmission unit 41.1, 41.2 comprises a camshaft 37, which is connected to the output shaft 36 for conjoint rotation. A cam disk 39.1 (second embodiment) or a cam disk 39.2 (third embodiment) is mounted on the camshaft 37 for conjoint rotation.

The cam disk 39.1 according to the second embodiment comprises a circumferential contour that varies along the circumference. In other words: The distance between the outer contour and the central axis DA of the cam disk 39.1 varies along the circumference thereof. Therefore, if viewed in a direction parallel to the central axis DA, the cam disk 39.1 does not have the shape of a circle, but instead the shape of a snail, for example. The circumference of the cam disk 39.1 comprises a segment 25 with a maximum radius, as well as an edge 26 at which the radius r changes abruptly, see FIG. 6.

The cam disk 39.2 according to the third embodiment comprises an end face with a varying surface contour. More precisely: The surface of the cam disk 39.2 that points toward the rod 4 is curved, for example steplessly rise. This surface is at a distance from a plane perpendicular to the axis of rotation DA, said distance varying across the surface.

The rod 4 is passed through a bracket 19. A tappet 60 is mounted on the end of the rod 4 that points toward the cam disk 39.1, 39.2. A compression spring 61 is supported against the wall 40 and strives to move the rod 4 toward the cam disk 39.1, 39.2 and thereby press the tappet 60 against the circumferential contour of the cam disk 39.1 (second embodiment) or against the surface contour of the cam disk 39.2 (third embodiment) and keep it in a contacting position. As a result, the tappet 60 is in continuous contact with the circumferential contour of the cam disk 39.1 or with the cam disk 39.2, even if the cam disk 39.1, 39.2 is being rotated about the axis of rotation DA.

In addition, in the exemplary embodiment, a perforated disk 43 with multiple cutouts is mounted on the camshaft 37 for conjoint rotation, see FIG. 5 and FIG. 7. A light barrier 44 is supplied with electrical energy by means of two electrical contacts 45.1, 45.2. A light source of the light barrier 44 emits a light beam. Depending on the rotational position of the perforated disk 43 and thus of the cam disk 39.1, 39.2, the emitted light beam passes through a cutout in the perforated disk 43 and impinges on a receiver of the light barrier 44, or is interrupted by the perforated disk 43. Therefore, thanks to the light source, the current rotational position of the cam disk 39.1, 39.2 can be measured. In the third embodiment, the cam disk 39.2 with the variable surface contour and the perforated disk 43 form a single component, which is mounted on the camshaft 37 for conjoint rotation.

Thanks to the perforated disk 43, the oscillating movement of the rod 4 can be controlled relatively reliably. The control makes it possible for a gas sample Gp to be drawn off by suction from the breath sample Ap, wherein the gas sample Gp that is drawn off by suction comprises exhaled air from at least one desired region of the test subject's respiratory system and is ideally completely free of exhaled air from at least one other region of the respiratory system. To achieve this aim, the motor 27 is actuated and moves the rod 4 in a controlled manner. The current rotational position of the cam disk 39.1, 39.2 determines the current position of the rod 4 and thus the current volume of the bellows 5.

As already explained, a fluid guide unit 71 connects the input unit 70 to the measurement chamber 3. A fluid connection comprising three segments 31, 15, 18 connected in series is provided in the interior of the fluid guide unit 71. A valve comprising a valve body 2 and a valve body seat 13 closes the fluid guide unit 71 or opens the latter, depending on whether the valve body 2 bears against the valve body seat 13 or a gap occurs. It is desired that the valve 2, 13 actually closes the fluid connection 31, 15, 18 in a fluid-tight manner and thus isolates the measurement chamber 3 from the surrounding environment if no gas sample Gp is to be drawn off by suction and the measurement chamber 3 is not to be flushed out either. This prevents a substance in the sensor from evaporating, which may be the case particularly with an electrochemical sensor, or conversely prevents an undesirable environmental influence from occurring on a sensor, in particular the ingress of particles.

However, the undesirable event may occur that particles get between the valve body 2 and the valve body seat 13 and thus the valve 2, 13 does not close fully in a fluid-tight manner, even if the valve body 2 bears against the valve body seat 13. It will be described below how the risk of this undesirable event occurring is reduced according to the disclosure.

The analyzer 100 additionally comprises a filter 23 in the interior of the fluid guide unit 71, namely at a position upstream of the valve 2, 13, i.e. between the input unit 70 and the valve 2, 13 if the input unit 70 is attached. The gas sample Gp, which is drawn off from the input unit 70 by suction, first flows through the filter 23 and then reaches the valve 2, 13. The filter 23 filters out, from the gas sample Gp flowing therethrough, all the particles which are larger than a specified upper limit or which have another specified property. This upper limit is predefined by the design of the filter 23 and is, for example, 2 μm.

In the exemplary embodiment, the filter 23 is located in the interior of the connecting piece 16 and also inside the connector 32. Other positions are also possible. The filter 23 is arranged so far away from the input unit 70 that the risk of the filter 23 being damaged by an external influence is relatively low. On the other hand, the filter 23 is positioned so far away from the measurement chamber 3 that, in every possible position of the rod 4, there is a gap between the valve body 2 and the filter 23.

Typically, the filter 23 comprises a filter element and a holder that surrounds and holds the filter element. The holder can be inserted into and removed from a corresponding receptacle in the main body of the analyzer 100. The gas sample Gp flows through the filter element. The filter element is electrostatically charged and comprises a nonwoven fabric, particularly a melt-blown nonwoven fabric. This embodiment leads to a filter with a relatively low pneumatic resistance and thus also a relatively low pressure drop across the filter 23. The nonwoven fabric or some other filter element of the filter 23 has a hydrophobic coating or is made of a hydrophobic material. This increases the reliability that moisture in the gas sample Gp will roll off the filter element and will not condense on the filter element, moisten the filter element, or even pass through the filter 23.

The filter 23 is inserted into a slot in the housing and can be replaced by access from outside.

In the exemplary embodiment, the gas sample Gp originates from a breath sample Ap provided by a test subject and therefore has a relatively high moisture content. An embodiment in which the filter element of the filter 23 is made of a hydrophobic material or at least has a hydrophobic coating has been described above. Compared to other possible implementations, this implementation reduces the risk of liquid droplets in the gas sample Gp damaging the filter element.

A different or additional possible measure is as follows: The filter 23 is heated, and can be heated in a contactless manner. Thanks to the heating, the temperature in a segment hS of the fluid guide unit 71 in which the filter 23 is located remains above the dew point of liquid in the breath. In many cases, this reliably prevents moisture from condensing on the filter 23.

Different implementations of a suitable heater are possible. In the exemplary embodiment, the heater used is located outside the fluid guide unit 71, including also outside the connector 32, and heats in a contactless manner a segment hS of the fluid guide unit 71, see FIG. 8. The schematically shown and electrically operated heater 24 comprises a light source, for example at least one LED, which emits warming electromagnetic radiation eS toward the filter 23. By way of example, the electromagnetic radiation eS heats the connector 32, and the heating of the connector 32 is transferred to the filter 23. The illustrated position of the light source 24 is to be understood merely as an example. Instead of a heater outside the fluid guide unit 71, use can also be made of a heating resistor inserted in the connector 32, in particular a heating coil.

In the exemplary embodiment shown, a schematically shown pressure sensor 46 repeatedly measures the pressure at a first measurement position MP.1 and optionally the pressure at a second measurement position MP.2. The first measurement position MP.1 is located downstream of the filter 23, for example in the fluid guide unit 71 or in or on the measurement chamber 3 or between the measurement chamber 3 and the suction unit 5, 6, 7, 27. The optional second measurement position MP.2 is located in the fluid guide unit 71 and upstream of the filter 23, i.e. between the filter 23 and the input unit 70.

A signal-processing control unit 60 receives a signal from the pressure sensor 46 and derives from the signal the temporal course of the pressure at the first measurement position MP.1 and optionally the temporal course of the pressure at the measurement position MP.2. If the pressure is measured at both measurement positions MP.1 and MP.2, the control unit 60 additionally derives the temporal course of the difference between the two pressures.

In one embodiment, the control unit 62 derives, from the temporal course of the pressure difference, the volume flow from the suction opening AO through the fluid guide unit 71 and thus through the filter 23 into the measurement chamber 3. A significant volume flow usually only occurs if the valve 2, 13 is open. The volume flow is caused by the suction unit 5, 6, 7, 27.

In one application, the control unit 62 derives, from the measured or otherwise determined volume flow, the volume of the gas sample Gp in the measurement chamber 3. It is known that volume is the integral over time and over volume flow. The time period over which integration is carried out is equal to the time period during which the suction unit 5, 6, 7, 27 sucks in the gas sample Gp and therefore during which the pressure at the measurement position MP.2 is lower than the pressure at the measurement position MP.1.

A further application of the pressure sensor 46 will be described below.

While a gas mixture flows through a mechanical filter 23, a pressure drop usually occurs across the filter 23. This pressure drop, i.e. the difference between the pressure upstream and the pressure downstream of the filter 23, can be considered with sufficient approximation as proportional to the volume flow of the gas mixture flowing through. This quotient is referred to as the pneumatic resistance of the filter 23. It is usually justified to assume that the pneumatic resistance does not depend significantly on the volume flow. If the volume flow through the filter 23 and the pressure drop across the filter 23 are known, the current pneumatic resistance of the filter 23 can be derived.

Inevitably, particles that the filter 23 filters out from the gas samples Gp flowing through settle on a surface of the filter 23. As a result, the pneumatic resistance of the filter 23 increases. If the measured current pneumatic resistance reaches an upper limit, the filter 23 should therefore be replaced. It will be described below how the control unit 62 determines the pneumatic resistance of the filter 23.

After a new filter 23 is inserted, it has an initial pneumatic resistance. This is usually predefined by the design of the filter. If the measured pneumatic resistance of the filter 23 is lower than the initial pneumatic resistance or even is equal to zero, this is an indication that no filter is inserted or that the filter 23 is inserted incorrectly or is defective.

The control unit 62 measures or determines the pressure drop across the filter 23 and the volume flow through the filter 23. The pressure drop and the volume flow occur in a time period during which the valve 2, 13 is open and during which the suction unit 5, 6, 7, 27 sucks in the gas sample Gp.

One embodiment for measuring the pressure drop and the volume flow has been described above. Said embodiment requires that both the pressure at the first measurement position MP.1 and the pressure at the second measurement position MP.2 are measured repeatedly. The embodiment described below does not require that the pressure at the second measurement position MP.2 be measured.

The pneumatic resistance of the fluid guide unit 71 is low compared to the pneumatic resistance of the filter 23. Therefore, if no filter 23 were present in the fluid guide unit 71, the following sequence would usually occur:
The drive 7, 27 is activated.
The volume of the bellows 5 is increased. At the same time, the valve 2, 13 is opened.
A gas sample Gp is sucked into the measurement chamber 3.
After a generally very short transient phase, the pressure in the measurement chamber 3 equals the pressure in the fluid guide unit 71, namely upstream of the filter 23. It is known that pressure propagates at approximately the speed of sound.

According to the disclosure, however, a filter 23 is present in the fluid guide unit 71. Therefore, at the latest after the end of the transient phase, a pressure drop in the fluid guide unit 71 is caused substantially by the filter 23.

In one embodiment, the control unit 62 determines a reference pressure at the first measurement position if the valve 2, 13 is closed and the drive 7, 27 is deactivated, for example as a function of a measured value from the pressure sensor 46. As explained above, the control unit 62 also determines the temporal course of the pressure at the first measurement position MP.1 while the valve 2, 13 is open and the gas sample Gp is being sucked in. From the reference pressure and the temporal course of the pressure, the control unit 62 derives the following information:
an average pressure drop across the filter 23 while the gas sample Gp is being sucked in and after the transient phase has elapsed, and
the time period and thus the time taken to suck in the gas sample Gp.

At the first measurement position MP.1, a negative pressure relative to the reference pressure usually only occurs in the time period during which the gas sample Gp is being sucked in.

In addition, the control unit 62 determines the volume of the gas sample Gp that is sucked in. In the exemplary embodiment, the gas sample Gp is sucked in by increasing the volume of the bellows 5. Usually, therefore, the volume of the gas sample Gp—after a transient phase—is equal to the difference between the maximum volume and the minimum volume of the bellows 5. This difference in volume is known from the geometry and design of the suction unit 5, 6, 7, 27 and is predefined.

As the volume flow through the filter 23, the control unit 62 uses the quotient of the volume of the gas sample Gp and the time taken to suck in the gas sample Gp.

The control unit 62 determines the current pneumatic resistance of the filter 23 as the quotient of the measured or determined pressure drop and the measured or determined volume flow. The control unit 60 determines the current pneumatic resistance repeatedly. For example, the control unit 62 determines the current pneumatic resistance again after the analyzer 100 has sucked in N gas samples since the last determination, where N>=1 is a specified number, or if the total summed volume of the gas samples since the last determination is greater than a specified upper limit.

The analyzer 100 generates a message in at least one form perceivable by a human if the pneumatic resistance of the filter 23 has reached a specified upper limit. This message contains information regarding the fact that the filter 23 needs to be replaced. The analyzer 100 causes this message to be output in at least one form perceivable by a human if the measured pneumatic resistance of the filter 23 has reached this limit. This message informs a user that the filter 23 should now be replaced.

In one embodiment, the analyzer 100 can ascertain whether or not a filter 23 is inserted. If no filter is inserted, the pneumatic resistance in the fluid guide unit 71 is significantly lower, in particular lower than the initial pneumatic resistance mentioned above. Or the analyzer 100 comprises a contact switch that is actuated by an inserted filter 23. Or the analyzer detects a confirmation by a user that a filter 23 has been inserted. In one embodiment, the control unit 62 prevents the suction unit 5, 6 from being activated and sucking in a gas sample if no filter 23 is inserted. The analyzer 100 then also generates a message to this effect.

### LIST OF REFERENCE SIGNS

1 hollow tip of the fluid guide unit 71, detachably connected to the input unit 70
2 valve body, mounted on the rod 4, belongs to the valve in the fluid guide unit 71
3 cylindrical measurement chamber, receives the gas sample Gp that is drawn off by suction, surrounded by the wall 40 and the cover plate 17, has the central axis MA
4 rod, is moved in the two directions R.1 or R.2 by the solenoid 7 or by the motor 27, increases the volume of the bellows 5 and moves the valve body 2 relative to the valve body seat 13
6 plate in the bellows 5, permanently connected to the sleeve 11 and thus to the rod 4
7 solenoid, can be activated and deactivated, after being activated moves the rod 4 relative to the fluid guide unit 71 and thus increases the volume of the bellows 5
8 fluid connection between the measurement chamber 3 and the interior of the bellows 5
9 frame of the main body of the analyzer 100
10 tubular, outflow-side connector, fastened to the wall 40
11 sleeve, connected to the rod 4
12 electrochemical sensor, comprises the electrodes 20 and 21 and the electrolyte 28
13 valve body seat 13 in the form of a sealing ring on the connecting piece 16, belongs to the valve in the fluid guide unit 71
14 sealing ring
15 segment in the connecting piece 16, belongs to the fluid guide unit 71 between the input unit 70 and the measurement chamber 3
16 tubular connecting piece of the fluid guide unit 71, connects the tip 1 to the connector 32, comprises the parts 16.1 and 16.2
16.1 smaller part of the connecting piece 16
16.2 larger part of the connecting piece 16
17 cover plate on the wall 40
18 segment beneath the measurement chamber 3, belongs to the fluid connection between the input unit 70 and the measurement chamber 3
19 bracket, through which the rod 4 is passed
20 measuring electrode of the sensor 12
21 counter-electrode of the sensor 12
22 connecting wire between the contact wires 33 and 34
23 electrostatically charged and/or mechanically acting filter in the segment 15 of the fluid connection between the surrounding environment and the measurement chamber 3
24 heater for the filter 23, comprises a radiation source in the form of an LED light, emits electromagnetic radiation eS
25 segment with maximum radius in the circumferential contour of the cam disk 39.1
26 edge in the circumferential contour of the cam disk 39.1
27 motor, rotates the output shaft 36 in the direction of rotation DR
28 ionically conductive electrolyte between the electrodes 20 and 21
29 measuring resistor in the connecting wire 22
31 segment in the tip 1, belongs to the fluid connection between the input unit 70 and the measurement chamber 3
32 inflow-side connector of the fluid guide unit 71, arranged on the wall 40
33 contact wire for the counter-electrode 21
34 contact wire for the measuring electrode 20
36 output shaft, rotated in the direction of rotation DR by the motor 27, rotates the cam disk 39.1, 39.2
37 camshaft
38 current intensity sensor, measures the intensity of the current flowing through the connecting wire 22
39.1 cam disk with eccentric outer contour, mounted on the camshaft 37 for conjoint rotation
39.2 cam disk with eccentric surface contour, mounted on the camshaft 37 for conjoint rotation
40 wall of the measurement chamber 3
41.1 transmission unit according to the second embodiment, in which the cam disk 39.1 has an eccentric outer contour
41.2 transmission unit according to the third embodiment, in which the cam disk 39.2 has an eccentric surface contour
42 reduction gear between the motor 27 and the output shaft 36
43 perforated disk, mounted on the camshaft 37 for conjoint rotation
44 light barrier comprising a light source and a receiver, comprises electrical contacts 45.1 and 45.2
45.1, electrical contact for the light barrier 44
45.2
46 pressure sensor, measures the pressure at the first measurement position MP.1 and optionally at the second measurement position MP.2
50 sensor arrangement comprising the measurement chamber 3 and the electrochemical sensor 12
60 tappet at the free end of the rod 4
61 compression spring, strives to push the rod 4 against the cam disk 39.1, 39.2
62 control unit
70 tubular input unit, has the inlet In, the outlet Out, the lateral surface M and the central axis EA
71 fluid guide unit, establishes a fluid connection between the suction opening AO in the input unit 70 and the measurement chamber 3, comprises the tip 1, the connecting piece 16 and the connector 32, has the longitudinal axis FA
100 analyzer, comprises the input unit 70, the fluid guide unit 71, the measurement chamber 3 in the wall 40, the sensor 12, the suction unit 5, 6, 7, 27 and the housing with the frame 9
AO suction opening in the lateral surface M of the input unit 70
Ap breath sample, is introduced into the input unit 70 through the inlet In by a test subject
Ap.r remainder of the breath sample Ap, which is not drawn off by suction and flows out of the input unit 70 through the outlet Out
Out outlet of the input unit 70
DA axis of rotation of the output shaft 36
DR direction of rotation, in which the motor 27 rotates the output shaft 36 and thus the cam disk 39.1, 39.2
EA central axis of the input unit 70, is perpendicular to the longitudinal axis FA of the fluid guide unit 71
FA longitudinal axis of the fluid guide unit 71, is perpendicular to the longitudinal axis EA of the input unit 70
In inlet of the input unit 70
eS electromagnetic radiation, emitted by the light source 24, heats the segment hS
Gp gas sample, which is drawn off (branched off) from the input unit 70 by suction and flows into the measurement chamber 3, is a portion of the breath sample Ap
hS segment of the fluid guide unit 71, heated by the emitted electromagnetic radiation eS
M tubular lateral surface of the input unit 70, has the suction opening AO
MA central axis of the cylindrical measurement chamber 3
MP.1 first measurement position: measurement position at which the pressure downstream of the filter 23 is measured, located in the fluid guide unit 71 or in or on the measurement chamber 3 or between the measurement chamber 3 and the suction unit 5, 6, 7, 27
MP.2 second measurement position: measurement position at which the pressure in the fluid guide unit 71 and upstream of the filter 23 is measured
O.a inlet to the measurement chamber 3
O.e outlet from the measurement chamber 3
r Varying radius 50 of the cam disk 39.1
R.1, R.2 anti-parallel directions, in which the rod 4 is moved

1. An analyzer for analyzing a gas mixture for a specified substance,
the analyzer comprising:
a main body,
a sensor arrangement in the interior of the main body,
a suction unit,
a fluid guide unit,
a tubular input unit,
a valve, and
a filter,
wherein the sensor arrangement comprises a measurement chamber and a sensor,
wherein the tubular input unit
comprises an inlet, an outlet, and a suction opening between the inlet and the outlet,
can be connected to the main body, and
is configured such that a gas mixture to be analyzed is introduced into the tubular input unit through the inlet and flows through the input unit towards the outlet,
wherein if the tubular input unit is connected to the main body, the fluid guide unit connects the suction opening to the measurement chamber,
wherein the suction unit is configured
to suck in from a gas mixture flowing through the input unit a gas sample from the input unit through the suction opening, and
to convey the sucked-in gas sample through the fluid guide unit into the measurement chamber,
wherein the sensor is configured to measure the concentration of the substance in the gas sample located in the measurement chamber,
wherein the valve
is movable back and forth between a closing end position and a releasing end position,
wherein the valve closes the fluid guide unit in the closing end position, and
opens the fluid guide unit in the releasing end position and
wherein the filter
is configured to filter particles out of the gas sample flowing through the filter,
is arranged in the fluid guide unit, and
is located between the suction opening and the valve when the input unit is connected to the main body.

2. The analyzer according to claim 1,
wherein the valve comprises a valve body and a valve body seat, and
the analyzer comprises a mechanical connecting element,
wherein the valve body bears against the valve body seat if the valve is in the closing end position, and a gap occurs between the valve body and the valve body seat if the valve is in the releasing end position, and
wherein the mechanical connecting element mechanically connects the valve body to the suction unit such that
while the suction unit sucks in a gas sample and conveys it into the measurement chamber,
the valve is moved from one end position to the other end position
the valve.

3. The analyzer according to claim 1,
wherein the analyzer comprises a heater,
wherein the heater is configured to heat a segment of the fluid guide unit, and
wherein the filter is located in the heated segment.

4. The analyzer according to claim 1,
wherein the input unit extends along a longitudinal axis, and the fluid guide unit extends along a longitudinal axis, and
wherein the two longitudinal axes enclose an angle of at least 60° between them.

5. The analyzer according to claim 1,
wherein the filter is electrostatically charged.

6. The analyzer according to claim 1,
wherein the filter comprises two layers,
wherein the two layers are arranged one behind the other and each have a plurality of openings,
wherein the openings are pores formed by fibers of the respective layer, and
wherein the openings are arranged offset from one another in such a way that a particle is deflected at least once on the way through the filter.

7. The analyzer according to claim 1,
wherein the analyzer comprises a pressure sensor,
wherein the pressure sensor is configured to measure the pressure at a first measurement position,
wherein the first measurement position is located in the fluid guide unit between the filter and the measurement chamber or on or in the measurement chamber or between the measurement chamber and the suction unit,
wherein the analyzer is configured
to measure the pressure at the first measurement position using a signal from the pressure sensor,
measure or capture the volume flow through the filter,
determine a current pneumatic resistance of the filter, and,
if the determined pneumatic resistance lies outside a specified value range,
generate a corresponding message and output it in at least one form perceivable by a human,
wherein the pneumatic resistance is the quotient of the pressure drop across the filter and the volume flow through the filter, and
wherein the analyzer is further configured to use
the measured pressure at the first measurement position and
the measured or captured volume flow through the filter
to determine the pneumatic resistance.

8. The analyzer according to claim 7,
wherein the pressure sensor is additionally configured to measure the pressure at a second measurement position,
wherein the second measurement position is located in the fluid guide unit and, if the input unit is attached, is located between the suction opening and the filter, and
wherein the analyzer is configured
to measure the pressure at the second measurement position using the signal from the pressure sensor, and
to determine the pressure drop across the filter and/or the volume flow through the filter using the two measured pressures.

9. The analyzer according to claim 7,
wherein the pressure sensor is configured to measure the pressure at the first measurement position repeatedly, and
the analyzer is configured
to determine a time period during which a negative pressure relative to a measured reference pressure occurs at the first measurement position,
to determine using the determined time period the time duration taken to suck in the gas sample,
to determine the volume of the gas sample sucked in, and
to determine the volume flow through the filter as the quotient of the volume and the determined time duration, and
wherein the analyzer is configured to determine a temporal course of the pressure at the first measurement position, and to use the temporal course of the pressure for determining the time period.

10. The analyzer according to any one of claims 7,
wherein the analyzer is configured to determine
if the determined pneumatic resistance of the filter is lower than a specified lower limit, and
to generate a message and cause this message to be output in at least one form perceivable by a human.

11. The analyzer according to claim 7,
wherein the sensor is configured to measure the concentration of alcohol as the substance in the gas sample in the measurement chamber.

12. A monitoring unit for monitoring an analyzer according to claim 1,
wherein the monitoring unit comprises a pressure sensor and a signal-processing evaluation unit,
wherein the pressure sensor is configured to measure a pressure at a first measurement position,
wherein the first measurement position is located in the fluid guide unit between the filter and the measurement chamber or on or in the measurement chamber or between the measurement chamber and the suction unit,
wherein the evaluation unit is configured
to determine the pressure at the first measurement position by using a signal from the pressure sensor,
determine the volume flow through the filter,
determine a current pneumatic resistance of the filter, and,
if the determined pneumatic resistance lies outside a specified value range,
generate a corresponding message and cause this message to be output in at least one form perceivable by a human,
wherein the pneumatic resistance is the quotient of the pressure drop across the filter and the volume flow through the filter, and
wherein the evaluation unit is further configured to use
the determined pressure at the first measurement position and
the measured volume flow through the filter
to determine the pneumatic resistance.

13. The monitoring unit according to claim 12,
wherein the pressure sensor is additionally configured to measure the pressure at a second measurement position,
wherein the second measurement position is located in the fluid guide unit and, if the input unit is attached, between the suction opening and the filter, and
wherein the evaluation unit is configured
to determine the pressure at the second measurement position using the signal from the pressure sensor, and
determine the pressure drop across the filter and/or the volume flow through the filter using the two determined pressures.
